(19) United States
(12) Patent Application Publication
(10) Pub. No.: US 2024/0409428 A1
(43) Pub. Date: Dec. 12, 2024

(54) RARE EARTH AUXILIARY AGENT AND PREPARATION METHOD THEREFOR

(71) Applicant: INNER MONGOLIA UNIVERSITY OF SCIENCE & TECHNOLOGY, Baotou, Inner Mongolia (CN)

(72) Inventors: LiTong MA, Baotou (CN); SongBo LI, Baotou (CN); Jun LI, Baotou (CN)

(21) Appl. No.: 18/697,354

(22) PCT Filed: Sep. 1, 2022
(86) PCT No.: PCT/CN2022/116440

§ 371 Date: Mar. 29, 2024

(30) Foreign Application Priority Data
Sep. 30, 2021 (CN) 202111164384.7

(51) Int. Cl. C01F 17/271; C01F 17/276; C12N 1/38

(52) U.S. Cl. CPC C01F 17/271; C01F 17/276; C12N 1/38; C01P 2004/61; C01P 2004/62; C01P 2006/20

(57) ABSTRACT

This present disclosure provides a rare earth additive and a method for preparing the rare earth additive to be used in production of clean energy or high-value chemicals, by which utilization of low-rank coal and biomass resources is achieved. The rare earth additive of the present disclosure is composed of rare earth chlorides, mixed rare earth chlorides, and rare earth nitrates, and can be used as additive for biochemical reactions between microorganisms and substances to be transformed, so as to improve the microbial activity in biochemical reactions. The rare earth additive promotes the transformation of low-rank coal (peat, lignite, sub-bituminous coal, weathered coal, coal gangue) and biomass into clean energy sources such as biomethane, biohydrogen, or bioethanol and high-value chemicals such as fulvic acid, water-soluble humic acid, benzoic acid, benzaldehyde, benzyl alcohol. The carbon reduction transformation of high-carbon resources such as low-rank coal and biomass may be achieved.

### CROSS-REFERENCE TO RELATED APPLICATIONS

This application claims priority to Chinese Patent Application No. 202111164384.7, entitled “Rare earth additive and preparation method therefor”, filed on Sep. 30, 2021, which is hereby incorporated by reference in its entirety.

### TECHNICAL FIELD

The present disclosure relates to the field of the application of rare earth resources and specifically relates to a rare earth additive. The present disclosure further provides a preparation method for the rare earth additive, which is suitable for industrial production.

### BACKGROUND

Rare earth elements consist of 17 elements, and the rare earth elements and the combination thereof have unique physicochemical properties. In the periodic table of chemical elements, “rare earth” collectively refers to 17 metal elements including lanthanides, scandium and yttrium. Rare earth elements are categorized into “light rare earth elements” and “heavy rare earth elements”. The light rare earths include lanthanum, cerium, praseodymium, neodymium, promethium, samarium, europium, and gadolinium; the heavy rare earths include terbium, dysprosium, holmium, erbium, thulium, ytterbium, lutetium, scandium, and yttrium. Such two kinds of elements differ in their electronic shell structures and physicochemical properties, as well as in their coexistence in minerals and their ionic radii.

Rare earth elements are typical metallic elements. Their reactivity of metal is second only to alkali metals and alkaline earth metals but is more reactive than other metallic elements. Among the 17 rare earth elements, metallic reactivity may increases from scandium, yttrium and up to lanthanum and decreases from lanthanum to lutetium. That is to say, lanthanum is the most reactive among the 17 rare earth elements. Rare earth elements may form chemically stable oxides, halides, and sulfides. Rare earth elements may react with nitrogen, hydrogen, carbon, and phosphorus and are soluble in hydrochloric acid, sulfuric acid, and nitric acid.

Rare earth elements, as non-renewable and scarce strategic resources, are known as the “vitamins in industry” and the “mother of new materials.” They are important strategic resources and are essential raw materials for industries such as electronic devices, aerospace, and new materials. Rare earth resources in China are characterized by favorable mineralization conditions, wide distribution, light rare earths in the north and heavy rare earths in the south, high content of valuable elements, and great comprehensive utilization value.

Currently, functional materials, such as rare earth permanent magnets, luminescent materials, hydrogen storage materials, and catalysts made from rare earth elements have become indispensable raw materials for advanced equipment manufacturing, new energy, and other high-tech industries. A Chinese patent CN200610057105.6 discloses a rare earth additive and calcium pyrophosphate containing the additive, as well as its preparation method. The main components include rare earths, dolomite powder, zeolite powder, and at least one of citric acid or citrates, with secondary components being magnesia powder and bentonite; by weight percentage, the proportions are: 10%-30% for rare earths, 10%-60% for dolomite powder, 10%-40% for zeolite powder, 1%-10% for citric acid or citrates, 5%-10% for magnesia powder, and 5%-10% for bentonite. This can be used to produce rare earth calcium pyrophosphate which, compared to regular calcium pyrophosphate, maintains the same levels of effective phosphorus, free acid, and moisture but has improved dispersibility. CN200710176223.3 discloses a fertilizer additive with rare earth slow-released and its preparation method. The additive contains at least one of porous mineral and rare earth salts that adhere to the surface of the mineral or inside the pores of the mineral. The porous minerals may include at least one of zeolites, perlite, diatomaceous earth, palygorskite, attapulgite, and dolomite, while the rare earth salts may include at least one of rare earth nitrates, rare earth chlorides, rare earth sulfates, rare earth acetates, and rare earth citrates. The rare earth may include at least one of lanthanum, cerium, prascodymium, and neodymium. The preparation method of the additive involves crushing the porous ore into particles with a size of 20-150 mesh, then immersing them in an aqueous solution of the rare earth salts and stirring the solution to make them even. The reaction is carried out at room temperature to 100° C. for 5-96 hours, after which the porous mineral particles that have adsorbed rare earth ions are taken out to be under drying process, so that the fertilizer additive with rare earth slow-released may be obtained. This patent is simple to be implemented, safe, highly effective, cost-effective, and suitable for large-scale production. This patent further discloses a fertilizer that contains the aforementioned additive.

The patents as mentioned above all involve the application of rare earth additives in fields such as petrochemicals and agriculture. However, there is few research on the application of rare earth elements in microbial biochemical conversion reactions.

Low-rank coal refers to coal at a stage of low metamorphism, which can be classified into low metamorphic bituminous coal and lignite based on the degree of coalification. A proven amount of reserved coal in China is 14,842.9 billion tons, of which an amount of low-rank coal is 59%, approximately 8,757.3 billion tons. Currently, over 90% of low-rank coal in China is used as fuel for electricity generation, industrial boilers, and domestic fuel under direct combustion, which renders a series of severe ecological and environmental pollution issues, and the oil, gas, and chemical resources contained within low-rank coal are wasted. In 2012, the contribution of coal in use to the annual average concentration of environmental PM2.5 in China was estimated to be 56%. Among this contribution, approximately 60% was generated by the direct combustion of coal, and about 40% was discharged by key industries using coal. Therefore, in order to solve the problem of coal combustion pollution, it is necessary to change the traditional way of mainly using coal as a fuel. A more rational approach is to use coal both as a fuel and as a raw material and implement graded transformation and tiered utilization on coal.

For low-rank coal, due to its low degree of coalification, high volatile matter content, and high moisture content, the efficiency of direct combustion or gasification is low. However, the chemical structure of its organic matter contains many side chains, and the elemental composition of the organic matter is high in hydrogen and oxygen content. If rare earth elements are utilized to help microbes in transforming low-rank coal and biomass, it can effectively improve material utilization efficiency and reduce environmental pollution and ecological problems.

### SUMMARY

The present disclosure is to provide a rare earth additive, composed of rare earth chlorides, mixed rare earth chlorides, and rare earth nitrates, and used as an additive for biochemical reaction between microbes and substances to be transformed. The rare earth chlorides consist of lanthanum chloride, cerium chloride, ytterbium chloride, and neodymium chloride. The mixed rare earth chlorides include at least one of lanthanum cerium chloride, praseodymium neodymium chloride, and samarium europium gadolinium chloride; the rare earth nitrates include at least one of lanthanum nitrate, cerium nitrate, ytterbium nitrate, and neodymium nitrate.

The prepared rare earth additive may significantly improve an efficiency of microbial biotransformation, promoting the transformation of low-rank coal and biomass into clean power sources such as biomethane, biohydrogen, and bioethanol, or into high-value chemicals such as fulvic acid, water-soluble humic acid, benzoic acid, benzaldehyde, and benzyl alcohol. This facilitates the carbon reduction transformation of high-carbon resources and reduces the consumption of high-carbon energy.

To achieve the aforementioned objectives, the present disclosure provides a rare earth additive, including in a unit of weight:
5-15 units of Lanthanum chlorides;
30-40 units of Cerium chlorides;
30-40 units of Ytterbium chlorides;
20-30 units of Neodymium chlorides;
1-5 units of Mixed rare earth chlorides;
1-5 units of Rare earth nitrates.

The rare earth chlorides such as lanthanum chloride, cerium chloride, ytterbium chloride, and neodymium chloride contain rare earth ions such as La³⁺ and Nd³⁺ which may improve the permeability of the cell membrane, allowing microbes to absorb and utilize nutrients better. Moreover, rare earth elements may influence microbial metabolic pathways and the enzymes involved in metabolism as cofactors, so that microbial metabolic capacity may be improved and the efficiency of microbial biotransformation may be improved.

The mixed rare earth chlorides may include lanthanum cerium chloride, praseodymium neodymium chloride, and samarium europium gadolinium chloride.

It may be empirically verified that lanthanum cerium chloride, praseodymium neodymium chloride, and samarium europium gadolinium chloride all have high catalytic activity, and a synergistic catalytic effect may be achieved in coordination with rare earth chlorides. Since the compounds found in rare earth mines are mixtures that are difficult to be separated, with the cost of separation thereof accounting for a significant proportion of a price of pure rare earth compounds, direct using of mixed rare earth chlorides as catalysts for biochemical reactions may have significant practical value in industry. This not only improves the catalytic effect in coordination with rare earth chlorides but also effectively reduces the cost of separation and production.

The rare earth nitrates mentioned in this present disclosure may include at least one of lanthanum nitrate, cerium nitrate, ytterbium nitrate, and neodymium nitrate.

There is papers in the prior art suggest that rare earth nitrates have an inhibitory effect on Escherichia coli and Staphylococcus aureus. However, it has found in actual research conducted for this present disclosure that rare earth nitrates are beneficial in improving the activity of anaerobic microorganisms and have a stimulating effect on growth of anaerobic microorganisms on some level. With this characteristic, rare earth nitrates are used in coordination with rare earth chlorides and mixed rare earth chlorides, the transformation efficiency of biochemical reactions with microorganisms involved may be improved. Moreover, rare earth nitrates may have a higher solubility compared to rare earth chlorides and are much easy to participate in reactions in a form of solution, with better activation effects. Better activation results may be achieved with a small amount of rare earth nitrates added.

The inventors have further verified that rare earth oxides and rare earth carbonates are insoluble in water, and cannot act on specific microorganisms or their microbial communities in biochemical reactions to promote chemical reactions with help of microorganisms, nor do they have an effect in synergy with rare earth chlorides.

In a preferred embodiment, the biochemical reaction may include a processes where specific microorganisms and their microbial communities may carry out certain chemical reactions by using microbial action, preferably carry out microbial transformation or hydrolysis or gas production reactions.

Biochemical reaction refers to chemical reaction carried out in biological organisms, specifically in plants and microorganisms. Biochemical reaction requires catalysis by enzymes and involves processes in which specific microorganisms and their microbial communities carry out certain chemical reaction with an action by microbial.

In a preferred embodiment, the substances to be transformed may include low-rank coal and biomass. Preferably, the low-rank coal may include peat, lignite, low metamorphic bituminous coal, weathered coal, and coal gangue.

In another preferred embodiment, the microorganisms may include hydrolytic bacteria, fermentative bacteria, hydrogen-producing bacteria, acetogenic bacteria, methanogenic bacteria, ethanologenic pseudomonads, and their communities.

In yet another preferred embodiment, the method for carrying out the biochemical reaction between the microorganisms and the substances to be transformed may include adding a rare earth additive to a reaction system of the substances to be transformed and water, and adding the microorganisms to carry out the biochemical reaction.

In a further preferred embodiment, the rare earth additive, the substances to be transformed, and water are configured into a reaction system at a mass/mass/volume ratio of (1-10): 100: (200-500) in use, and then microorganisms may be inoculated to improve the biochemical reaction.

In a preferred embodiment, the volume ratio of the reaction system to the microbial donor ranges is (0.1-10): 1. Preferably, the volume ratio of the reaction system to the microbial donor is (1-2): 1, and more preferably, the volume ratio of the reaction system to the microbial donor is 1:1.

In another preferred embodiment, the microbial donor has a moisture content greater than 40%. Preferably, the moisture content of the microbial donor is 40-98%.

In yet another preferred embodiment, the microbial donor is activated sludge. Activated sludge is a collective term for a community of microorganisms and the organic and inorganic substances they adhere to, and may be obtained from the semi-solid residual sludge discharged by wastewater treatment plants.

Aerobic microbial communities can be eliminated from the activated sludge under sealed anaerobic conditions after diluting the activated sludge with water in a laboratory container, to acclimatizing the activated sludge to become anaerobic activated sludge, which may include obligate anaerobic bacteria and their communities, as well as facultative anaerobic bacteria and their communities. With abundant microbial communities in anaerobic activated sludge, such as hydrolytic bacteria, fermentative bacteria, hydrogen-producing bacteria, acetogenic bacteria, methanogenic bacteria, and ethanologenic pseudomonads, the microorganisms may rapidly reproduce and ferment during the fermentation process. The rate of anaerobic fermentation and the utilization rate of raw materials may be improved, and high yields of methane and hydrogen, as well as high raw material utilization rates may be achieved. The residual sludge may be acclimatized to become anaerobic activated sludge without the need for specific culture media or nutrient solutions, to obtain a microbial community rather than a single bacterium. It may be feasible for the microbial biotransformation with the addition of additives as needed, so as to generate clean energy or high-value chemicals.

With additives for biochemical reaction prepared by the present disclosure for dealing organic waste from sewage treatment plants, it is possible to achieve a rough and easy acclimatization process, and significantly reduce production costs. A considerable cost advantage may be achieved in industrial-scale operations and waste emissions may be reduced, with resource utilization improved.

Another objective of this present disclosure is to provide a rare earth additive. First, rare earth chlorides, mixed rare earth chlorides, and rare earth nitrates are mixed thoroughly, then subjected to a two-step crushing process to prepare an ultrafine powder of the rare earth additive, which is then sealed and set aside for use.

The method for preparing the rare earth additive in the present disclosure is simple and convenient, with no additional restrictions and requirements on production conditions or operators, and process is simplified, and especially suitable for large-scale industrial production.

To achieve the aforementioned objectives, the present disclosure provides a method for preparing a rare earth additive, which includes the following steps:
Step S1: performing evenly mixing, crushing, and screening with 40-200 mesh standard sieve on rare earth chlorides, mixed rare earth chlorides, and rare earth nitrates to obtain rare earth additive powder in 40-200 mesh;
Step S2: performing ultrafine crushing on the rare earth additive powder for 3-30 minutes to obtain ultrafine powder of the rare earth additive.

In a preferred embodiment, in step S1, a high-speed multifunctional crusher is used for performing crushing, and then the screening is performed with 400 mesh standard sieve; in step S2, an air separation ultrafine crusher is used to perform ultrafine crushing.

In a preferred embodiment, a particle size of the ultrafine powder is 0.214 μm-10 μm.

A rare earth composition is mechanically crushed to crush large mineral particles into even and fine powder, then an ultrafine crusher is used to break the material particles and crush them to particles in a size of 10 μm or less, which makes significant changes in the microstructure and surface chemical properties of the rare earth additive, and the ultrafine powder of the rare earth composition may have characteristics such as increased specific surface area, porosity, and surface energy, so that material may have unique physicochemical properties such as high fluidity, high solubility, and high adsorption, which may improve the permeability of microbial cell membranes in the biochemical reaction system, allow microbes to better absorb and utilize nutrients in the fermentation reaction system to meet the nutritional needs for microbial growth and reproduction, further to promote microbial metabolism, and clean energy and high-value chemicals may be obtained. Practical verification has shown that if the particle size of the rare earth composition is greater than 10 μm, its ability to catalyze microbial reactions significantly decreases, and its chemical transformation capacity weakens. If the particle size of the rare earth composition is less than 0.214 μm, the cost of crushing increases without further improvement in catalytic capability. Therefore, considering cost and effectiveness, the particle size of the ultrafine powder is limited to 0.214 μm-10 μm.

Compared with prior art, rare earth additive and its preparation method according to the present disclosure have the following advantages:

1. The rare earth additive described in the present disclosure is precisely formulated with rare earth compounds as the effective components. The biocatalytic performance of the rare earth additive acts on low-rank coal which has organic matter and humic acids as main components so as to break specific chemical bonds in the low-rank coal, dissociate corresponding functional groups, and transform them into the carbon source needed by microbes for the conversion for biomethane, biohydrogen, or bioethanol, to obtain the corresponding clean energy. The rare earth additive described in the present disclosure may be used to break the specific chemical bonds of humic acids contained in low-rank coal, dissociate corresponding functional groups, and transform them into high-value chemicals such as fulvic acid, water-soluble humic acids, benzoic acid, benzaldehyde, benzyl alcohol, to realize transformation of high carbon resources in low-rank coal with carbon reduced or no carbon, which is of great significance for the high-value utilization of low-rank coal resources in China.

2. The rare earth additive for biochemical reactions described in the present disclosure leverages the synergistic interactions among components of rare earth additive to ensure sustained activity in various reaction systems and adaptability to complex environmental factors. The rare earth additive is highly targeted and has pronounced effects which may accelerate specific reactions under mild conditions and specially for generating clean energy sources such as biohydrogen, biomethane, or bioethanol, or high-value chemicals such as fulvic acid, water-soluble humic acids, benzoic acid, benzaldehyde, benzyl alcohol with low-rank coals such as peat, lignite, sub-bituminous coal, weathered coal, and coal gangue as raw materials.

3. The preparation method of the rare earth additive for biochemical reactions described in the present disclosure may be obtained only by thoroughly mixing the components and then crushing them into an ultrafine powder. Such preparation method is simple, suitable for large-scale industrial production, and can achieve large-volume continuous production. The rare earth additive made by such method may be directly turned into a practical commercial product after simple packaging.

### BRIEF DESCRIPTION OF THE DRAWINGS

FIG. 1 is a curve diagram shows comparison between methane productions of embodiment 1 of the present disclosure and comparative experiment 1.

FIG. 2 is a curve diagram shows comparison between hydrogen productions of embodiment 2 of the present disclosure and comparative experiment 2.

### DETAILED DESCRIPTION

Unless specifically stated, the technical means used in the embodiments are conventional means well known to those skilled in the art, and the raw materials used are commercially available products.

Unless otherwise specified, various raw materials, reagents, instruments, and equipment used in this present disclosure can be purchased in the market or prepared by existing means.

In this present disclosure, weight units may be well-known weight units such as μg, mg, g, kg, or multiples thereof, such as 1/10, 1/100, 10 times, 100 times thereof.

In the embodiments of the present disclosure, the microbial donor used is the excess activated sludge discharged from the Baotou city sewage treatment plant, with a moisture content of 60%.

Detection means:

Methane production is determined daily and in total by an AMPTSII device to obtain a daily methane production and a total methane production. Biomethane produced by a fermentation unit first passes a NaOH absorption unit so that only CH4 enters a gas volume measurement unit. PH value is determined by using a Leici PHS-25 PH meter. Dehydrogenase activity is determined by using spectrophotometry. Concentration of acetic acid is determined by using an Agilent-1260 Infinity High-Performance Liquid Chromatograph, with an Agilent Hi-Plex H (7.7 mm×300 mm, 8 μm) as liquid chromatography column. A differential detector is used as a detector. A column temperature is set at 60° C., 0.005 mol/L sulfuric acid solution is used for mobile phase, and a flow rate is set as 0.5 mL/min.

Hydrogen production is determined by the water displacement method.

### Embodiment 1

The rare earth additive described in the embodiment includes the following components by mass content:
8 units of lanthanum chloride;
40 units of cerium chloride;
30 units of ytterbium chloride;
20 units of neodymium chloride;
1 unit of samarium europium gadolinium chloride;
1 unit of lanthanum nitrate.

The preparation method for the rare earth additive is as follows: mixing the above-mentioned raw materials of rare earth additive thoroughly, crushing the mixed raw materials and performing screening thereon with a 400 mesh sieve to obtain powder of rare earth additive. Then, ultrafine grinding may be performed on the powder of the rare earth additive for 3-30 minutes to obtain ultrafine powder of rare earth additive.

60 g of lignite coal may be crushed to be of 100 mesh, which are then added to a anaerobic reaction bottle of 500 mL, into which 200 mL of anaerobic activated sludge may be added then, with a total of 3 g of the rare earth additive weighed and mixed in proportion to be added thereafter, and pure water to be filled until the total reaction system reaches 450 mL. The pH is adjusted to be 7.0 and a methane production experiment is conducted at a high temperature of 50° C. The amount of gas generated thereby would be the most on the 17th day, which is 176.7 mL, and the cumulative total amount of gas generated over 35 days is 1151.6 mL.

### Comparative Experiment 1:

Anaerobic activated sludge+lignite coal without adding a rare earth additive.

200 mL of anaerobic activated sludge and 60 g of lignite coal may be crushed to be of 100 mesh without adding any rare earth additive, and pure water is filled therein until the total reaction system reaches 450 mL. Using the same method as in embodiment 1, the pH is adjusted to be 7.0 and a methane production experiment is conducted at a high temperature of 50° C. The amount of gas generated thereby would be the most on the 20th day, which is 144.2 mL, and the cumulative total amount of gas generated over these days is 822.5 mL.

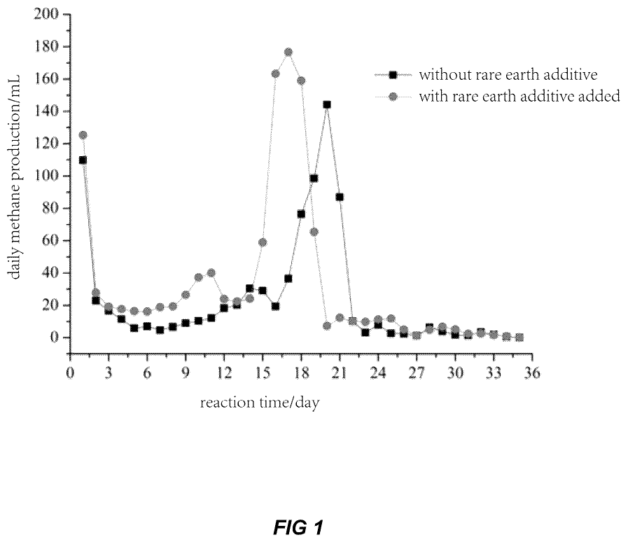

The curve diagram showing comparison between methane productions of embodiment 1 of the present disclosure and comparative experiment 1 is shown in FIG. 1.

### Embodiment 2

The rare earth additive described in this embodiment includes the following components by mass content:
5 units of lanthanum chloride;
40 units of cerium chloride;
30 units of ytterbium chloride;
20 units of neodymium chloride;
4 units of praseodymium neodymium chloride;
1 unit of ytterbium nitrate.

The preparation method for the rare earth additive is same as in Embodiment 1.

60 g of lignite may be crushed to be of 100 mesh, which is then added into a anaerobic reaction bottle of 500 mL, and then 200 mL of anaerobic activated sludge that has been heated at 100° C. for 30 minutes to kill methanogenic bacteria may be added therein, with a total of 3 g of the rare earth additive weighed and mixed in proportion to be added thereafter, and pure water to be filled until the total reaction system reaches 450 mL. The pH is adjusted to be 7.0 and a hydrogen production experiment is conducted at 50° C. The amount of hydrogen generated thereby would be the most on the 1st day, which is 93.7 mL, and decreased gradually thereafter. The cumulative total amount of hydrogen generated from 1st day to the day when the generation of hydrogen is stopped is 262.1 mL. Comparative Experiment 2:

Anaerobic activated sludge with methanogenic bacteria killed after being heated at 100° C. for 30 minutes and lignite, without rare earth additives added.

There are only pure water, 200 mL of anaerobic activated sludge with methanogenic bacteria killed after being heated at 100° C. for 30 minutes, and 60 g of lignite crushed to be of 100 mesh in the reaction system, and pure water is filled up thereafter without adding rare earth additives until the total reaction system reached 450 mL. The pH was adjusted to 7.0, and the hydrogen production experiment is conducted at 50° C. The amount of hydrogen generated thereby would be the most on the 1st day, which is 33.0 mL, and decreased gradually thereafter. The cumulative total amount of hydrogen generated from 1st day to the day when the generation of hydrogen is stopped is 94.8 mL.

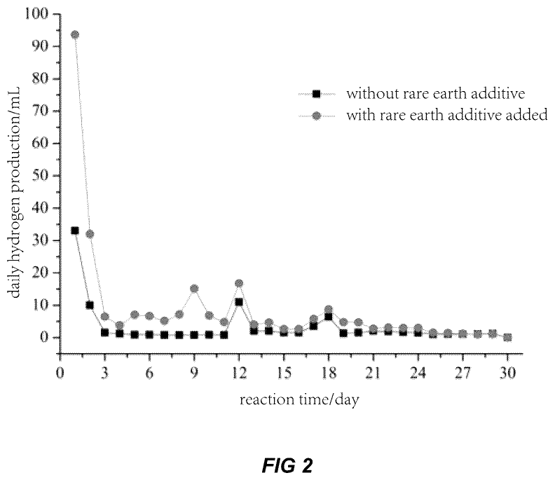

The curve diagram showing comparison between hydrogen productions of embodiment 2 of the present disclosure and comparative experiment 2 is shown in FIG. 2.

### Embodiment 3

The rare earth additive described in this embodiment includes the following components by mass content:
5 units of lanthanum chloride;
40 units of cerium chloride;
30 units of ytterbium chloride;
20 units of neodymium chloride;
4 units of lanthanum cerium chloride;
1 unit of neodymium nitrate.

The preparation method of the rare earth additive is same as in embodiment 1.

30 g of lignite may be crushed to be of 100 mesh, which is added to a reaction bottle of 500 mL, and 200 mL of activated sludge and 200 mL of pure water may be added therein. Then, a total of 1.5 g of the rare earth additive weighed and mixed in proportion to be added thereafter, and an experiment of degradation of lignite humic acid is conducted. The concentration of humic acid reaches a peak on the 4th day, which is 545.60 mg/L, the concentration of benzoic acid reaches a peak on the 4th day, which is 0.5491 mg/L, the concentration of lignite benzyl alcohol reaches a peak on the 3rd day, which is 1.3367 mg/L, and the concentration of benzaldehyde reaches a peak on the 4th day, which is 1.4605 mg/L.

Comparative Experiment 3: Anaerobic Activated sludge+Lignite.

There are only 200 mL of pure water, 200 mL of activated sludge, and 30 g of lignite crushed to be of 100 mesh without rare earth additives added. The experiment for the degradation of lignite humic acid was carried out. The concentration of humic acid reaches a peak on the 4th day, which is 435.16 mg/L, the concentration of benzoic acid reaches a peak on the 4th day, which is 0.3382 mg/L, the concentration of lignite benzyl alcohol reaches a peak on the 3rd day, which is 1.1367 mg/L, and the concentration of benzaldehyde reaches a peak on the 4th day, which is 1.3634 mg/L.

The detailed exemplary embodiments described above for the present disclosure are intended for illustration and description. These descriptions are not intended to limit the present disclosure to the specific details as disclosed, and it is obvious that many changes and variations can be made in light of the above teachings. The purpose of selecting and describing these exemplary embodiments is to explain the specific principles of the present disclosure and their practical implementations so that those skilled in the art may implement and utilize various exemplary embodiments of the present disclosure, as well as various alternatives and modifications. The scope of the present disclosure is intended to be defined by the claims and their equivalents.

1. A rare earth additive, characterized in that, the rare earth additive is composed of rare earth chloride, mixed rare earth chloride, and rare earth nitrate, and used as additive for biochemical reaction between microbes and substances to be transformed;
wherein the rare earth chloride is composed of lanthanum chloride, cerium chloride, ytterbium chloride, and neodymium chloride,
the mixed rare earth chloride comprises at least one of lanthanum cerium chloride, praseodymium neodymium chloride, and samarium europium gadolinium chloride;
the rare earth nitrate comprises at least one of lanthanum nitrate, cerium nitrate, ytterbium nitrate, and neodymium nitrate.

2. The rare earth additive according to claim 1, characterized in that, the rare earth additive composed of, in a unit of weight,:
5-15 units of lanthanum chlorides;
30-40 units of cerium chlorides;
30-40 units of ytterbium chlorides;
20-30 units of neodymium chlorides;
1-5 units of mixed rare earth chlorides;
1-5 units of rare earth nitrates.

3. The rare earth additive according to claim 1, characterized in that, the substances to be transformed comprises low-rank coal and biomass.

4. The rare earth additive according to claim 3, characterized in that, the low-rank coal comprises peat, lignite, low metamorphic bituminous coal, weathered coal, and coal gangue.

5. The rare earth additive according to claim 1, characterized in that, the rare earth additive, the substances to be transformed, and water are configured into a reaction system at a mass/mass/volume ratio of (1-10): 100: (200-500) in use, and then microorganism is inoculated, so as to improve biochemical reaction.

6. A preparation method of the rare earth additive according to claim 1, characterized in that, comprising following steps:
Step S1: performing evenly mixing, crushing, and screening with 40-200 mesh standard sieve on rare earth chlorides, mixed rare earth chlorides, and rare earth nitrates to obtain rare earth additive powder in 40-200 mesh;
Step S2: performing ultrafine crushing on the rare earth additive powder for 3-30 minutes to obtain ultrafine powder of the rare earth additive.

7. The preparation method of the rare earth additive according to claim 6, characterized in that, a particle size of the ultrafine powder is 0.214 μm-10 μm.
